(19) United States
(12) Patent Application Publication
(10) Pub. No.: US 2022/0130518 A1
(43) Pub. Date: Apr. 28, 2022

(54) SYSTEMS AND METHODS FOR MENTAL HEALTH ASSESSMENT

(71) Applicant: Tibi Health, Inc., San Francisco, CA (US)

(72) Inventors: Simal OZEN IRMAK, San Francisco, CA (US); Utku IRMAK, San Francisco, CA (US)

(21) Appl. No.: 17/516,477

(22) Filed: Nov. 1, 2021

Related U.S. Application Data
(63) Continuation of application No. PCT/US2021/055825, filed on Oct. 20, 2021.
(60) Provisional application No. 63/104,364, filed on Oct. 22, 2020.

(51) Int. Cl. G16H 20/70; G16H 10/20; G16H 50/30

(52) U.S. Cl. CPC G16H 20/70; G16H 50/30; G16H 10/20

(57) ABSTRACT

The present disclosure provides methods and systems for monitoring mental health of a subject. The methods and systems may comprise: (a) collecting data attributable to the subject at different time points; (b) providing the data to a computer system programmed with a machine learning algorithm, where the machine learning algorithm may process the data and may determine a status of mental health of the subject; and (c) providing the status of mental health of said subject to a recipient. The data may be derived from answers to inquiries provided on a plurality of requests individualized to the subject.

### CROSS-REFERENCE TO RELATED APPLICATIONS

This application is a continuation of International Application No. PCT/US2021/055825, filed Oct. 20, 2021, which claims the priority and benefit of U.S. Provisional Application No. 63/104,364, filed on Oct. 22, 2020, both of which are incorporated herein by reference in their entirety.

### BACKGROUND

Mental health can be examined in various ways. For example, in clinical psychiatry a clinical assessment process gathers information from different sources including evaluating a subject's appearance, behavior, speech, psychomotor activity, mood, thought process, judgment, and cognitive functions. This information can be used to formulate diagnosis or treatment plans. Objective diagnostic and prognostic measures can play an important role in health care. However, communication with the subject has remained an important source of information in diagnosing and treating mental disorders.

### SUMMARY

Recognized herein is a need for early detection of mental disorders (e.g., perinatal mood and anxiety disorders). Barriers to timely access to mental health care include (but are not limited to) shortage of mental health providers, underestimation of its priority, poor screening and diagnostic tools in primary care setting to navigate patients to appropriate mental health resources, lack of awareness of a subject that they may be in need of mental healthcare, lack of knowledge of a subject in how to access appropriate mental health care, lack of time of a subject, stigma in seeking mental health care and insufficient awareness of mental disorders. After these barriers are overcome, in-person psychotherapeutic or psychiatric assessments pose their own limitations. A subject's recall-bias when providing detailed history about their emotional states at different points in times may affect the accuracy and quality of care provided. These limitations may pose critical delay and discontinuity in mental care of the subject, which can cause significant and life-threatening conditions. For example, perinatal monitoring and screening of a subject for mental disorders is critical and time-sensitive for both subject and newborn wellbeing.

A mental health status of a subject can be monitored over time to identify change in patterns and behavior to predict a risk of developing a mental health condition such as depression. For example, a longitudinal data of social, behavioral, biological, affective/cognitive, mood, psychomotor activity, experiential, sociodemographic, or medical health markers of a subject in real-world conditions can be collected. Temporal trends within this data may be recognized, including using artificial intelligence (AI), and based on a predictive model an impending health risk for a subject can be identified. For example, a propensity of a mental health risk score can be calculated for a subject on an ongoing basis. Subsequently, based on the risk calculated at various time points, an effective feedback or an actionable recommendation can be provided.

In one aspect, a method for monitoring mental health of a subject is provided. The method may comprise: (a) collecting data attributable to the subject at different time points, where the data can be derived from answers to inquiries provided on a plurality of requests individualized to the subject; (b) providing the data to a computer system programmed with a machine learning algorithm, which machine learning algorithm processes the data and determines a status of mental health of the subject; and (c) providing the status of mental health of the subject to a recipient.

In some embodiments, the subject is perinatal (i.e., trying, expecting or postpartum). In some embodiments, the subject is not perinatal. In some embodiments, the subject is a parent (e.g., biological mother, biological father, intended parent, adoptive parent or foster parent). In some embodiments, the subject is not a parent (e.g., egg donor, gestational carrier or an individual going through fertility treatment).

In some embodiments, the status of mental health comprises a status of depression, a status of anxiety, a status of mood, a status of obsessive compulsive disorder, a status of psychosis, a status of suicidality, a status of distress, a status of stress, a status of bipolar disorder, a status of baby blues, a status of post-traumatic stress disorder, a status of eating disorder, a status of sleep disorder or any combination thereof. In some embodiments, the status of depression comprises a status of perinatal-associated depression.

In some embodiments, the machine learning algorithm processes the data and determines a risk of a mental condition in the subject. In some embodiments, the machine learning algorithm determines a risk score for the mental condition in the subject. In some embodiments, the mental health status is predictive.

In some embodiments, the status of mental health of the subject is provided to the recipient on a report. In some embodiments, the method further comprises providing a recommendation associated with the status of mental health of the subject to the recipient. In some embodiments, the recommendation comprises a recommendation for a therapy or sources of education associated with the status of mental health.

In some embodiments, the method further comprises alerting the recipient to a behavioral risk associated with the status of the mental health. In some embodiments, the behavioral risk is a risk of suicide of the subject, risk of infanticide being committed by the subject, risk of developing perinatal depression, risk of developing anxiety, risk of developing obsessive compulsive disorder, risk of developing psychosis, risk of developing distress, risk of developing stress, risk of developing bipolar disorder, risk of developing baby blues, risk of developing post-traumatic stress disorder, risk of developing sleep disorder, risk of developing eating disorder, or any combination thereof. In some embodiments, the behavioral risk is a perinatal behavioral risk.

In some embodiments, the recipient is the subject. In some embodiments, the recipient is not the subject. In some embodiments, the answers to the inquiries are provided by the subject. In some embodiments, the answers to the inquiries are not provided by the subject. In some embodiments, the answers to the inquiries are provided via automatic data extraction. In some embodiments, the answer to the inquiries are provided by another subject different from the subject.

In some embodiments, at least two requests of the plurality of requests comprise at least one different inquiry. In some embodiments, at least one request of the plurality of requests is individualized to the subject based on answers to another request that precede the at least one request. In some embodiments, a request of the plurality of requests comprises a single inquiry. In some embodiments, (a) further comprises collecting the data attributable to the subject over a time period of at least two weeks.

In some embodiments, the inquiries include an inquiry associated with health, an inquiry associated with weight, an inquiry associated with social interactions, an inquiry associated with a physiologic state, an inquiry associated with cognitive affective state, an inquiry associated with environmental conditions, an inquiry associated with healthcare utilization, an inquiry associated with mood, an inquiry associated with exercise level, an inquiry associated with nutrition, an inquiry associated with appetite, an inquiry associated with psychomotor activity, an inquiry associated with expressive behaviors (e.g., facial expression, body language or speech), an inquiry associated with tobacco usage, an inquiry associated with alcohol consumption, an inquiry associated with social support, an inquiry associated with sleep, an inquiry associated with social and behavioral markers of health, an inquiry associated with depression, an inquiry associated with anxiety, an inquiry associated with distress, an inquiry associated with stress, an inquiry associated with obsessive compulsive behavior, an inquiry associated with daily experiences, an inquiry associated with childcare, an inquiry associated with breastfeeding, an inquiry associated with parenting, an inquiry associated with prior mental health problems, an inquiry associated with medical history, an inquiry associated with familial medical history, an inquiry associated with substance abuse, an inquiry associated with clinical diagnosis, an inquiry associated with socio-demographic state, an inquiry associated with family structure, an inquiry associated with household conditions, an inquiry associated with exposure to domestic violence or sexual assault, an inquiry associated with living conditions, an inquiry associated with subject characteristics (e.g., race, ethnicity), education, or income level, or any combination thereof.

In some embodiments, the method further comprises collecting additional data attributable to the subject (i) via a device configured to monitor one or more health or wellness markers associated with the subject (ii) from an individual. In some embodiments, the device is a mobile electronic device. In some embodiments, the individual is a parent of the subject, a friend of the subject, a partner of the subject or a household member of the subject. In some embodiments, the individual is a care-provider. In some embodiments, the care-provider is a health-care provider, a lactation consultant, a psychotherapist, a psychiatrist, a physical therapist, a social worker, health support professional (e.g., birth doula, postpartum doula) or an exercise and wellness professional (e.g., prenatal yoga instructor, postpartum yoga instructor). In some embodiments, the one or more health or wellness markers is sleep, an activity level, an exercise level, a psychomotor activity level, speech, nutrition, appetite, weight, an emotional state, social relations, a bonding of the subject with the subject's children, or any combination thereof.

In some embodiments, the method further comprises providing the additional data to the machine learning algorithm which machine learning algorithm processes the additional data to determine the status of mental health of the subject. In some embodiments, the method further comprises collecting additional data obtained from one or more medical or clinical tests conducted with respect to the subject. In some embodiments, the one or more medical tests comprise a blood test, saliva test, a screening test, a clinical diagnostic test, a test under Diagnostic and Statistical Manual of Mental Disorders (DSM) guidelines, a biometric test, an activity test, a sleep test, a mental health test, psychoanalysis or a behavioral test. In some embodiments, the method further comprises collecting additional data obtained from one or more medical or clinical diagnosis made with respect to the subject. In some embodiments, in (a), the status of the mental health of the subject is unknown. In some embodiments, the subject has a mental state corresponding to a level of less than, at or greater than a screening or diagnostic-threshold on the Edinburgh Postnatal Depression Scale (EPDS), Patient Health Questionnaire (PHQ) or General Anxiety Disorder (GAD) screening tool. In some embodiments, the subject has a mental state corresponding within 10% above or below a screening or diagnostic-threshold value on the Edinburgh Postnatal Depression Scale (EPDS), Patient Health Questionnaire (PHQ) or General Anxiety Disorder (GAD) screening tool.

In another aspect, a method of generating a machine learning tool is provided. The method may comprise (a) providing data attributable to a subject to a machine learning algorithm, where a mental health status of the subject is undetermined; (b) determining the mental health status of the subject; (c) generating an identifier that identifies the data attributable to the subject as attributable to the mental status of the subject; and (d) producing the machine learning tool by training the machine learning algorithm with the identifier.

In some embodiments, (d) comprises producing the machine learning tool by training the machine learning algorithm with the data attributable to the subject. In some embodiments, the method further comprises repeating (a)-(c) for data attributable to a plurality of subjects. In some embodiments, the method further comprises in (c) generating a plurality of identifiers that identify the data attributable to the plurality of subjects as attributable to mental health statuses of subjects of the plurality of subjects. In some embodiments, the method further comprises in (d), producing the machine learning tool by training the machine learning algorithm with the plurality of identifiers. In some embodiments, (d) comprises producing the machine learning tool by training the machine learning algorithm with the data attributable to the plurality of subjects. In some embodiments, (b) comprises clinically determining the mental health status of the subject. In some embodiments, (d) comprises clinically determining the mental health status of the subject.

In some embodiments, the method further comprises using the machine learning tool to assess an individual mental health status in an individual. In some embodiments, the machine learning tool predicts mental status of an individual with at least about 80% greater accuracy than the machine learning algorithm does prior to (d). In some embodiments, the data attributable to the subject is data obtained at a plurality of time points. In some embodiments, the method further comprises repeating (a)-(c) for additional data attributable to the subject and the data attributable to the subject obtained at a later time point from the subject, and where, in (a), the additional data is provided to the machine learning tool produced in (d). In some embodiments, the method further comprises, in (c), generating a time dependent identifier that identifies the additional data as attributable to a later mental health status of the subject at the later time point. In some embodiments, the method further comprises producing a further trained machine learning tool by training the machine learning tool with the time dependent identifier. In some embodiments, the machine learning tool comprises a database. In some embodiments, the identifier is stored in the database. In some embodiments, a plurality of identifiers are stored in the database. In some embodiments, the plurality of identifiers comprises a plurality of time-dependent identifiers. In some embodiments, the machine learning tool comprises a sequence model, where the sequence model predicts an identifier for additional data provided to the machine learning tool.

In some embodiments, the subject is perinatal (trying, expecting or postpartum). In some embodiments, the subject is not perinatal. In some embodiments, the subject is a parent (e.g., biological mother, biological father, adoptive parent, foster parent, etc.). In some embodiments, the subject is not a parent (e.g., egg donor, gestational carrier, an individual going through an infertility treatment).

In some embodiments, the status of mental health comprises a status of depression, a status of anxiety, a status of mood, a status of obsessive compulsive disorder, a status of psychosis, a status of suicidality, a status of distress, a status of stress, a status of bipolar disorder, a status of baby blues, a status of post-traumatic stress disorder, a status of eating disorder, a status of sleep disorder, or any combination thereof. In some embodiments, the status of depression comprises a status of perinatal-associated depression.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a flow chart of an example of a mental health monitoring procedure.

FIG. 2 schematically illustrates an example of a system for a computer-implemented algorithm.

FIG. 3 schematically illustrates an example of training a machine learning algorithm.

FIG. 4 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “subject” generally refers to a human subject. A subject can be, for example, a parent (e.g., mother, father, step-parent, adoptive parent, etc.), or a person associated with a parent (e.g., family member of such a parent, friend of such a parent, etc.). A subject can be a prospective parent who is using fertility treatment or trying to conceive. A subject may be a person who is planning to become a parent. A subject may be an expecting or postpartum mother or father. A subject may have 1, 2, 3, 4, 5, 7, 8, 9 10, or more children. A subject may be a person other than the parent or person associated with a parent subject who is assisting the parent subject in providing answers and/or communicating according to the systems and methods provided herein. A subject may be male or may be female. A subject may also identify as male, female or both male and female.

As used herein, the term “recipient” generally refers to an entity that receives a result comprising mental health status according to the systems and methods provided herein. The recipient can be the subject. The recipient can be a person(s) other than the subject. The recipient can be a family member or a friend of the subject, a helper, a medical professional, a medical provider, a hospital representative, or a health care clinic representative.

As used herein, the term “inquiry” or “inquiries” generally refers to a message, a reminder, a hard copy, a questionnaire, a survey, a test or the like that poses a question, or a set of questions to collect information about a subject or a condition associated with a subject. In some cases, the inquiry or inquiries can be self-initiated by a subject (e.g., to provide additional information). In some cases, (upon the subject's permission) the inquiry or inquires can be automatically (i.e., passively) collected from the electronic and/or wearable devices the subject uses.

As used herein, the term “individualized” generally refers to a question or an inquiry designed based in part on information attributable to a subject. An individualized inquiry or questionnaire can be different for each subject. An individualized inquiry or questionnaire can be designed to collect information specific about a subject in a way that may or may not be present in a population of subjects.

As used herein, the term “status of mental health” or “mental health status” generally refers to a mental condition of a human subject. A status of mental health of a subject may comprise a status of healthy behavior, positive mood, or healthy mental condition, or a status of unhealthy behavior, negative mood, or unhealthy mental condition of a subject. A status of mental health may comprise, for example, a status of depression, a status of anxiety, a status of suicidal thoughts, a status of sadness, a status of harmful thoughts, a status of obsessive-compulsive behavior, a status of psychosis, a status of perinatal depression and anxiety, a status of eating disorder, a status of sleep disorder, or a status of other mood or anxiety problem.

As used herein, the term “automatic data extraction” generally refers to collecting information from a device or an application without a need for human supervision or interaction. Data generated by a wearable sensor or an application running on a smart device can be requested automatically. An automatic data extraction can be performed using an application programming interface (API). An automatic data extraction can be performed in predefined intervals, at predefined time point, or periodically at random times.

As used herein, the terms “continuous,” or “substantially continuous” generally refer to an action or condition that periodically or irregularly repeats, sometimes over very short periods. Continuous may refer to an event, happening, condition, or action that is very frequent at very short time intervals. A continuous event or condition may be present for a long period of time compared to the short intervals. The long period of time may be at least a minute, an hour, a day, a week, a month, a year, or more. The short interval may be at most a minute, an hour, a day, a week, or a year. An action may be performed every second for a year, or every minute for a week, or two times a day for 6 months, etc.

Provided herein is a method to monitor a status of mental health of a subject in a period of time. A mental disorder can initiate, develop, or progress over time in a subject without obvious or recognizable symptoms. By monitoring a status of mental health of a subject, the method disclosed herein can recognize or detect a change in a subject's behavior; the change in behavior trends may be used for early detection of a mental disorder. The early detection of the mental disorder can warn the subject to seek help (e.g., from a psychiatrist or family members) in time and receive treatment if necessary. The early detection can also be used to apply preventative measures that may prevent a subject from developing a more severe mental disorder.

The monitoring of a subject's mental health status may be performed using multiple strategies, which may include asking a subject directly, or monitoring a subject's physical activities such as sleeping patterns or psychomotor activity levels such as speech patterns or expressive behaviors such as facial expressions or virtual activates such as activities on social media. Medical history of the subject (e.g., previous mental health assessments, blood test reports, etc.) can also be used to collect information about the subject. The monitoring system can communicate questions to collect information about the subject or behaviors thereof. These questions can be personalized for the subject. The subject or another user (e.g., family members of a subject or a healthcare provider) can provide answers to set of questions asked by the monitoring system. In some cases, an answer to an inquiry is provided by the subject. Alternatively, an answer to an inquiry is provided by a person other than the subject. In some cases, an answer to an inquiry is provided via completing a survey or typing free-text or recording an audio clip or recording a video clip or communicating via text, audio or video with others. In some cases, an answer to an inquiry is automatically (i.e., passively) collected from the electronic and/or wearable devices the subject uses.

Monitoring methods described herein can also make use of an artificial intelligence algorithm (AI) as described herein to process the data collected by monitoring the subject. The AI (e.g., a machine learning algorithm) can recognize trends (e.g., normal trend) in a subject's behavior and even predict a future behavior based on a trend that has been detected. The AI can also establish a trend for behaviors associated with mental health disorders (e.g., different disorders or different levels of a disorder) using historic data generated from individuals with one or more mental disorders. The AI can be configured to detect a deviation from a normal trend in a subject that may not be aligned with a future behavior predicted by the AI or a similarity in the behavior of a subject to a trend associated with a mental health disorder. Subsequently, the AI determines a status of mental health for a subject based on the behavior trends and/or the detected changes or similarities.

The mental health status can be determined for a subject in a period of time that the subject is being monitored regularly or intermittently. A report of the status of health can be generated and sent to a subject or another recipient (e.g., a family member of a subject or a healthcare provider). Based on the report, the subject or the other recipient, or both can take appropriate actions. One or more actions can also be provided by the monitoring system which may be reported as suggestions to the user or the other recipient. A recipient may also diagnose a mental disorder or choose a method of treatment of a disorder for the subject based on the report.

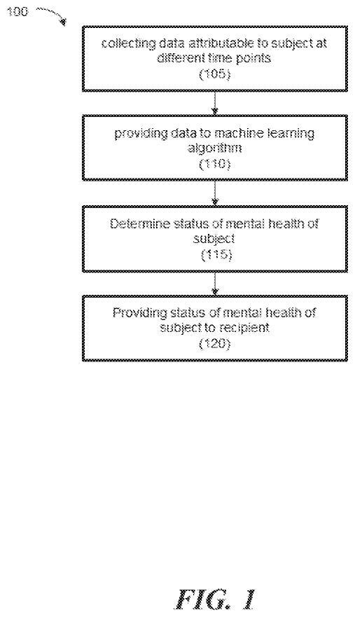

FIG. 1 shows a flow chart of an example method 100 for determining a status of mental health of a subject. At an operation 105, a method 100 may comprise collecting data attributable to a subject at various time points. At an operation 110, the method 100 may comprise providing the collected data to a machine learning algorithm. At the operation 110, the method 100 may further comprise processing the collected data by the machine learning algorithm comprising determining a set of parameters related to a status of mental health of the subject. At an operation 115, the method 100 may comprise determining a status of mental health of the subject based at least in part on the processed data from the operation 110. At an operation 120, the method 100 may comprise providing a status of mental health of the subject to a recipient. The recipient may be the subject or a person other than the subject.

Although the above operations show a method 100 of determining a status of mental health of a subject, in accordance with some embodiments, many variations can be implemented as described herein. The operations may be completed in any order. Operations may be added or deleted. Some of the operations may comprise sub-operations. Operations may be repeated as often as appropriate. One or more operations may be repeated before or after one or more operations may be performed. For example, in some embodiments, an operation 105 may be performed before the operation 110 and after operation 115, in order to collect more information attributable to a subject to improve an accuracy of a determination of a mental health status of the subject.

An aspect of the disclosure provides a method for monitoring mental health of a subject. The method may comprise: (a) collecting data attributable to the subject at different time points, where the data may be derived from answers to inquiries provided on a plurality of requests individualized to the subject, (b) providing the data to a computer system programmed with a machine learning algorithm; the machine learning algorithm may process the data and determine a status of mental health of the subject; and (c) providing the status of mental health of the subject to a recipient.

In some cases, data can be obtained directly or indirectly from a subject. Data may be obtained directly from a subject through standard and/or custom-designed (e.g., individualized to a subject) inquiries (e.g., questionnaires or surveys). Data may be obtained directly from inquiries self-initiated by the subject. An inquiry can be communicated with a subject through a communication device (e.g., a cellphone or a computer) using a communication tool (e.g., an application, web-based survey, e-mail, phone messaging such as SMS or MMS). An inquiry can be communicated with a subject in an in-person meeting (e.g., physical meeting or virtual meeting). Data may be obtained indirectly from a subject through devices (e.g., tablet or smart-phone) or sensors (e.g., heart-rate sensor). A sensor may be embedded in a wearable (e.g., smart watch or an activity tracker) that a subject may use, or it may be a stand-alone sensor (e.g., a blood oxygen sensor, blood pressure monitoring sensor, sleep monitoring sensor, or an electroencephalogram). The data obtained from a subject may include a comprehensive set of health markers comprising disease biomarkers, biometrics, cognitive state biomarkers, psychomotor activity and expressive behavior biomarkers (e.g., speech, facial expressions or body language), medical or non-medical health markers.

In some cases, a questionnaire may comprise an Edinburgh Postnatal Depression Scale (EPDS) comprising a depression screening tool. The EPDS may be used for a preconception period, prepartum period and/or postpartum period. In some cases, a questionnaire may comprise a version of Patient Health Questionnaire (e.g., PHQ-9, or PHQ-2). In some cases, a questionnaire may comprise a version of Generalized Anxiety Disorder (GAD) questionnaire (e.g., GAD-7, GAD-2) or any other available standardized questionnaire. In some cases, a questionnaire may comprise a pregnancy experiences questionnaire comprising a survey information on: a trimester (e.g., a first trimester, a second trimester, or a third trimester), conception, a period prior to conception or current physical and mental health history, a survey associated with a birthing experience, key health statistics regarding a delivery of a newborn, a health status of the newborn, information associated with a postpartum period (e.g., first month, second month, third month, fourth month, fifth month, sixth month, seventh month, ninth month, tenth month, twelfth month, second year, third year, fourth year, fifth year, or longer) or a period in between any two time periods mentioned herein. In some cases, a questionnaire may comprise a pregnancy experiences questionnaire comprising one or more surveys associated with developmental milestones, sleeping, feeding or communicative behaviors of the newborn. In some cases, a questionnaire may comprise a fertility, adoption, loss, pre-conception, prepartum or postpartum mood experiences questionnaire.

In some cases, data can be obtained from an individual other than the subject (e.g., a healthcare provider, a clinician, a family member of the subject or a friend of the subject). In some cases, the person may be designated or authorized by the subject to communicate information about the subject. In some cases, data may be collected from a database comprising data attributable to a subject such as an electronic health record database (EHR). In some cases, the information obtained about the subject may comprise information about a person or persons associated or related to the subject (e.g., family history, family health status, or friends). The information obtained about the subject may comprise, for example, data related to the subject's social behavior, relations, social activities, or virtual social activities (e.g., social media activities). In some cases, the data obtained may comprise any type of data pertinent to a subject's behavioral, cognitive or affective state, health, wellbeing, social interactions, environmental conditions, healthcare utilizations, medical and clinical data (e.g., data obtained from blood, saliva, other invasive or non-invasive medical tests and screenings), biometrics, psychomotor activity data, expressive behavior data (e.g., speech or body language), activity, sleep, or data relevant to the subject's family members (e.g., household members, subject's child or newborn) or living conditions. For example, sleeping data associated with the subject's newborn child may be obtained and used in the method described herein. In some cases, a medical test may comprise a blood test, saliva test, a screening test, a clinical diagnostic test, a test under diagnostic and statistical manual of mental disorders (DSM or DSM-V) guidelines, a biometric test, an activity test, a sleep test, a mental health test, a cognitive test, psychoanalysis or a behavioral test.

In some cases, the data collected from the subject may comprise data associated with social data, behavioral data, biological data, affective or cognitive data, psychomotor activity data, expressive behavior data (e.g., speech, body language or facial expressions), experiential data, sociodemographic data, and medical health marker data. For example, the social data may comprise data related to inner social support (e.g., familial support) or external social support (e.g., external support). The behavior data may comprise data related to sleep (e.g., duration of sleep, sleep fragmentation, sleep states, sleep frequency, intensity of sleep), physical activity, psychomotor activity, verbal and non-verbal expressive state (e.g., sentiment, content or pitch energy of verbal communications, facial expressions, body language), nutrition eating behavior, appetite or physical ability (e.g., upper-body strength, lower-body strength). Non-limiting examples of the biological data may comprise a heart rate, weight of the subject, body mass index (BMI), presence of chronic health conditions. Non-limiting examples of the health data may comprise any physical or nonphysical symptoms being experienced by the subject (e.g., pain, headache, or urinary incontinence). Non-limiting examples of the medical data may comprise a prior history or an existing state of mental health of the subject, any medical history of the subject, medical utilization data (e.g., date of medical visits, types of medical utilization, frequency of medical visits), or medication data. The medical data may further comprise data associate with obstetrical or gynecological data such as data related to conception experience, pregnancy experience, birthing experience, or prior fertility experience. In some cases, the data related to a prior fertility experience relates to an infertility treatment (e.g., intrauterine insemination (IUI) or in vitro fertilization (IVF)).

The data associated with an affective state (mental state) of a subject may be collected using standardized questionnaires or custom-designed questionnaires. Data associated with an affective state (mental state) may comprise overall mood, depressive state, bipolar state, anxiety level, cognitive state (e.g., neurocognitive state), obsessive-compulsive behavior, intrusive thoughts, psychotic state, sleep-wake state, or stress level (e.g., mental stress or emotional stress). In some cases, the data collected from the subject may comprise data associate with experiential or conditional situation of a subject comprising a living condition, an employment status, or a past or current life stressor (e.g., death of a loved one (including pregnancy loss), single parenting, domestic violence, pandemic conditions). The socio-demographic data may comprise, for example, age, ethnicity, race, discrimination (including, for example, perceived discrimination), income (e.g., salary, wage, or income level), or residential address.

In some cases, the sleep quality and sleep quantity data may be used to predict a mental health status of a subject. For example, perinatal period may be associated with significant sleep problems such as sleep deprivation and excessive sleep fragmentation. In some cases, sleep disturbances (e.g., sleep deprivation, sleep architecture anomalies (e.g., presence, order and duration of sleep cycles), disturbances in facilitation or continuation of sleep) may be associated with an affective disorder. In some cases, sleep deprivation is used to predict a mental health disorder. In some cases, depression is associated with deviation from the normal sleep patterns. For example, difficulties in falling asleep or failing to maintain sleep can also be a sign of elevated anxiety.

In some cases, a physical activity change is associated with a mental health status. During a perinatal period, physical activity or exercise level of a subject may decrease. In some cases, a lack of physical activity or a decrease in physical activity may be associated with depressive mood or fatigue. In some cases, decreased physical activity may be coupled with social isolation, which together can increase the risk of a subject developing a mental disorder.

In some cases, a change from normal weight of a subject, BMI, or body composition may be associated with a mental health status of a subject. For example, during the perinatal period a redefined degree change in the subject's weight, BMI or body composition may be expected. Excessive weight gain or weight loss may be determined and be associated with a mood or anxiety disorder. In some cases, a rapid and unintentional weight loss during the early postpartum period may be a strong sign for anxiety. In some cases, a gradual weight change (e.g., increase in weight, decrease in weight) during the postpartum period may be associated with depression.

In some cases, the heart rate or other cardiac measurements (e.g., pulse wave velocity) is collected to determine a mental health status of a subject. An increased level of anxiety or stress can be predicted using data associated with the cardiac measurements. In some cases, the cardiac measurements are used in combination with other markers of anxiety to predict a change in the anxiety level of a subject.

In some cases, the data associated with a subject's mood, happiness, satisfaction, expectations, disappointments or concerns are combined with other health markers to determine a health status of the subject. In some cases, the health (or wellness) marker comprises sleep, an activity level, an exercise level, a psychomotor activity level, nutrition, weight, appetite, an emotional state, social relations, expressions and/or a bonding of a subject (e.g., parent, grandparent) with children (e.g., newborn child). In some cases, the data can be used to determine an interventional strategy.

In some cases, the subject is pregnant. Alternatively, the subject may not be pregnant. A non-pregnant subject can be a woman, a man, a family member of an individual who is expecting a child, may have a newborn, adopting, fostering or assisting intended parents. In some cases, the subject may be parent, an expectant parent (e.g., perinatal), an intended parent, a foster parent or an adoptive parent. The parent may be a male, female, both, neither or a different gender. The expectant parent may be pregnant. The expectant parent may be receiving or subjected to fertilization treatments. For example, an expectant parent may be receiving in vitro fertilization (IVF), hormone therapy, or undergoing a procedure (e.g., surgery) associated with fertilization (e.g., reverse sterilization surgery). In some cases, the subject is a postpartum parent. In some cases, the subject may not be a parent. In some cases, the subject is an individual close to a parent or an expectant parent. For example, a subject may be a grandparent, a partner, a family member of a parent, etc.

In some cases, collecting data attributable to the subject may comprise obtaining data at different time points. For example, the same type of data may be collected at different time points. In some cases, different types of data may be collected at different time points. In some cases, data may be collected intermittently (e.g., random time point) or in predefined intervals (e.g., scheduled time points). A predefined interval to collect data may comprise one or more times per minute, per hour, per day, per week, per month, or per year. Alternatively, data may be collected substantially continuously. A frequency of collecting data may depend on the type of data (e.g., inquiry-based data, health care data, data obtained directly, or data obtained indirectly).

Directly obtained data may be collected less frequently than data obtained indirectly. For example, questionnaires may be sent to a subject or an individual other than the subject in the beginning of the monitoring and once every week, once every two weeks, once a month, once every two months, once every six months, or a frequency in between any two frequencies mentioned herein, thereafter. In some other cases, indirectly obtained data such as data from devices (e.g., tablet or smart-phone) or sensors (e.g., heart-rate sensor) may be collected at a higher rate. For example, an indirectly obtained data may be collected one or more times every minute, every hour, every day, every two days, every week, every month, or at a rate in between any two rates mentioned herein. Data may be collected substantially automatically (e.g., without a human person involved) using an application programming interface (API). For example, the subject may allow the method described herein to obtain data automatically from one or more data generating applications (e.g., social media applications or sleep monitoring applications) on the subject's device (e.g., smart phone, smart watch, sensor, etc.).

The subject may be monitored for a period of time using the method described herein. The period of monitoring the subject may be from about a day to about any number of years. The monitoring period may be about 1 day to about 10 days, about 7 days to about 30 days, about 10 days to about 90 days, about 30 days to about 120 days, about 80 days to about 270 days, about 175 days to about 365 days, about 300 days to about 700 days, about 350 days to about 900 days. In some cases, the period of monitoring may be least about: 5 days, 10 days, 15 days, 30 days, 45 days, 60 days, 100 days, 150 days, 200 days, 350 days, 365 days, 500 days, 600 days, 700 days, 800 days, 900 days, or more. In some cases, the period of monitoring may be most about: 900 days, 800 days, 700 days, 600 days, 500 days, 400 days, 365 days, 350 days, 300 days, 250 days, 200 days, 150 days, 100 days, 60 days, 45 days, 30 days, 15 days, 10 days, 5 days, or less. Data may be collected from the subject substantially continuously during the period of monitoring.

In some cases, an inquiry (e.g., a questionnaire) may be provided (e.g., through a request) two or more times (e.g., a plurality of times) to a subject or an individual other than the subject. For example, to validate a result or to track a change in a subject's health, behavior, relationships, etc. In some cases, at least 2, 3, 4, 5, 6, 7, 10, 20, 30, 40, 100, 200, 300, 400, 500, 1000, 2000, or more inquiries may be provided to a subject (e.g., the subject, or a person(s) other than the subject). In some cases, a new inquiry (e.g., a questionnaire) may be generated to collect new data attributable to the subject. For example, to follow up with a previous inquiry, to collect new data, or to track a change in a subject's health, behavior, relationships, etc. In some cases, an inquiry (e.g., a questionnaire) may be individualized to a subject. An inquiry may be individualized based on, at least in part on data obtained from the subject prior to requesting (e.g. communicating) the inquiry with the subject or an individual other than the subject. For example, based partially on data from the subject's EHR, an inquiry related to a subject's physical or mental health may be individualized to the subject. In some cases, an individualized inquiry may be generated for the subject based in part on a change in the subject's data (e.g., a change in a trend in the data or a new trend being detected in the data). Alternatively, an inquiry (e.g., a questionnaire) may be individualized to the subject based partially on answers provided to another inquiry (or request for information).

In some cases, a plurality of requests may be sent to a recipient (e.g., the subject, or a person(s) other than the subject). The request may comprise one or more inquiries. In some cases, multiple requests may be sent comprising one inquiry. In some cases, a request may comprise one inquiry. In some cases, at least 2, 3, 4, 5, 6, 7, 10, 20, 30, 40, 100, 200, 300, 400, 500, 1000, 2000, or more requests may be sent to a recipient during a period of monitoring a subject.

In some cases, an inquiry comprises an inquiry associated with health, an inquiry associated with weight, an inquiry associated with social interactions, an inquiry associated with a physiologic state, an inquiry associated with expressive behaviors (e.g., speech, facial expression or body language), an inquiry associated with psychomotor activity level, an inquiry associated with cognitive (e.g., neurocognitive) or affective state, an inquiry associated with environmental conditions, an inquiry associated with healthcare utilization, an inquiry associated with mood, an inquiry associated with exercise level, an inquiry associated with nutrition, an inquiry associated with appetite, an inquiry associated with tobacco usage, an inquiry associated with alcohol consumption, an inquiry associated with social support, an inquiry associated with sleep, an inquiry associated with social and behavioral markers of health, an inquiry associated with depression, an inquiry associated with anxiety, an inquiry associated with distress, an inquiry associated with stress, an inquiry associated with obsessive compulsive behavior, an inquiry associated with psychosis, an inquiry associated with suicidality, an inquiry associated with daily experiences, an inquiry associated with childcare, an inquiry associated with breastfeeding, an inquiry associated with parenting, an inquiry associated with prior mental health problems, an inquiry associated with medical history, an inquiry associated with familial medical history, an inquiry associated with substance abuse, an inquiry associated with clinical diagnosis, an inquiry associated with socio-demographic state, an inquiry associated with family structure, an inquiry associated with household conditions, or any combination thereof.

In some cases, a mobile platform is used to collect the data from the subject. The mobile platform may also store the collected data. For example, the subject may be asked periodically to provide information associated with the subject comprising physical activity, sleeping behavior, eating behavior, expressive behavior, psychomotor behavior, mood level, or other health markers (e.g., through online survey, text message, etc.). In some cases, data can be automatically collected (e.g., disclosed data, or consented data) through the usage of a device (e.g., a smart phone or a wearable device) or an API thereof (e.g., HealthKit on iOS operated phones). Data collection and/or communication with the subject may be performed using a webpage (e.g., using a computer, a laptop, or a desktop) or an app. Data collection, storage, and/or communication with the subject may follow rules and regulations associated with medical data (e.g., HIPAA). The collected data, generated data (e.g., data generated in communication) may be stored on a cloud storage (e.g., Amazon web services (AWS)).

The method described herein may further comprise providing the data to a computer system programmed with a machine learning algorithm. In some cases, the AI-based model (e.g., a machine learning, a trained machine learning algorithm, a machine learning tool) may receive one or more reference data sets associated with healthy individuals (e.g., determined by a clinician as mentally healthy) and/or individuals with known mental disorders (e.g., individuals that have been clinically diagnosed with a mental health disorder). In some cases, the AI-based model (e.g., a machine learning, a trained machine learning algorithm, a machine learning tool) may receive one or more of other reference data sets associated with clinical mental health guidelines such as the diagnostic and statistical manual of mental disorders (DSM-5). The AI model may determine the mental health status or a risk of mental disorder in the subject partially based on data (e.g., association, correlation, regression or temporal trends in data) in the reference data sets.

The machine learning algorithm can process the data and determine a status of mental health of the subject. In some cases, any combination of data, as described herein, or a trend in the data (e.g., a temporal trend, a dynamic trend) may be used to assess the subject's mental health status (e.g., cognitive, behavioral or affective state). In some cases, an associate risk (e.g., probability score or risk score) of a mental disorder may be determined (e.g., calculated or predicted) in the subject using an AI-based model (e.g., a machine learning, a trained machine learning algorithm, a machine learning tool). A status of mental health of the subject may comprise presence or absence of a mental health disorder. For example, a mental disorder risk score can be determined using the AI-based model based, at least in part, on data collected historically from the subject (e.g., a portion of or an amalgamation of past data) or a trend in the data (e.g., established by the AI). A portion of the historic data obtained from the subject may comprise the latest or the last data collected from the subject.

A status of mental health in the subject may comprise a status of depression, a status of anxiety, a status of mood, a status of obsessive compulsive disorder, a status of psychosis, a status of suicidality, a status of distress, a status of stress, a status of bipolar disorder, a status of baby blues, a status of post-traumatic stress disorder (PTSD), a status of eating disorder, a status of sleep disorder, or any combination thereof. In some cases, the status of health may comprise a status of perinatal mood and anxiety disorders (PMAD) comprising perinatal depression, perinatal anxiety, perinatal psychosis, perinatal bipolar disorder, perinatal obsessive-compulsive disorder or perinatal post-traumatic stress disorder. PMAD may also comprise scary or intrusive thoughts. In some cases, the status of depression is a status of perinatal-associated depression. The status of the mental health of a subject may comprise a change in a mental status of a subject. For example, the status of mental health of a subject may comprise an elevated mental disorder or condition associated to a mental disorder (e.g., an elevated depression, elevated suicidal tendencies, or an increased bipolar behavior.)

The method described herein may provide the status of mental health of the subject to a recipient. The status of mental health of the subject, determined using the AI model (e.g., a machine learning algorithm), can be provided to a recipient. In some cases, the mental health status of a subject is predictive. The status of mental health of the subject provided to a recipient may comprise a behavioral risk. In some cases, the recipient may comprise the subject or an individual other than the subject. The recipient may comprise a health care provider, a family member (e.g., a parent, a partner, a husband, a wife, a domestic partner), a friend, a health and wellness professional or a clinician (e.g., a psychotherapist, a counselor, a psychologist, a nurse, etc.)

A mental disorder risk score may comprise a probability of developing a mental disorder in a subject associated with a status of mental health of a subject. The behavioral risk may comprise a probability associated with a risk of suicide of the subject, a risk of infanticide being committed by the subject, a risk of developing perinatal mood and anxiety disorder (PMAD), a risk of developing depression, a risk of developing anxiety, a risk of developing obsessive compulsive disorder, a risk of developing psychosis, a risk of developing distress, a risk of developing stress, a risk of developing bipolar disorder, a risk of developing baby blues, a risk of post-traumatic stress disorder, or any combination thereof. In some cases, the behavioral risk may comprise a perinatal behavioral risk. A mental disorder risk score may comprise a combined probability of developing two or more mental disorders in a subject associated with a status of mental health of a subject.

The method described herein may further comprise communication with a recipient. In some cases, communication with a recipient may be through an application (e.g., web-based application, android application or iOS application). The application may comprise a dashboard or notification system. For example, an application may comprise synchronous or asynchronous text-based communication (e.g., text messaging, SMS, or electronic mail), voice-based communication (e.g., voice messaging or voice calls), or multimedia communication (e.g., multimedia messaging services (MMS) or video calls). The recipient comprising the subject or an individual other than the subject (e.g., someone assigned or authorized by the subject, a family member, a guardian of the subject, a clinician, or a healthcare provider) may be contacted or communicated with using communication methods described herein.

In some cases, the method described herein may further comprise providing to a recipient one or more recommendations (e.g., behavioral intervention, seeking mental health council, or a treatment regimen). The recommendation may be based in part on the status of mental health of the subject. The recommendation may be made directly to a subject or a clinician. The recommendation may comprise a set of behaviors or actions to help the subject to regain a previous mental health status (e.g., a healthy pattern or trend that was previously observed in a subject). The recommendation may comprise an alert or a warning to help a subject identify a behavior or trend which may be associated with mental disorder. The recommendation may be transmitted to or communicated with a subject via a message, a note, a graphical message, a voice, a sound, or video. In some cases, a recommendation may comprise a recommendation for a therapy or sources of education associated with said status of mental health. In some cases, a recommendation may comprise a personalized recommendation. A personalized recommendation may be at least in part based on a status of mental health of the subject, a data collected attributable to the subject, or a combination thereof. A personalized recommendation may comprise an associated time period (days, weeks, months, etc.) A personalized recommendation may further comprise an actionable suggestion. A non-limiting example of the actionable suggestion may comprise watching a video on a topic relevant to the status of mental health of the subject, joining a peer-support group, or meeting with a healthcare provider.

Another aspect of the disclosure provides a method of generating a machine learning tool; the method comprises: (a) providing data attributable to a subject to a machine learning algorithm, wherein a mental health status of the subject is undetermined; (b) determining the mental health status of said subject; (c) generating an identifier that identifies the data attributable to the subject as attributable to the mental status of the subject; and (d) producing the machine learning tool by training the machine learning algorithm with the identifier.

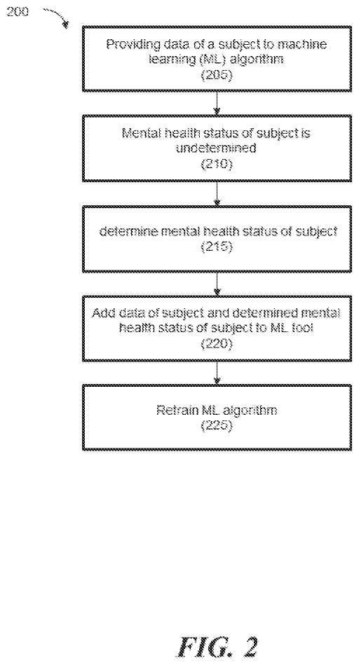

FIG. 2 shows a flow chart of an example method 200 for generating a machine learning tool, in accordance with some embodiments. At an operation 205, a method 200 may comprise providing data attributable to a subject to a machine learning (ML) algorithm. At an operation 210, the method 200 may comprise determining a mental health status of a subject as undetermined. At an operation 215, the method 200 may comprise determining a mental health status of the subject. In some embodiments, the operation 215 may comprise collecting additional data attributable to the subject. In some embodiments, the additional data may be provided to the ML of operation 205. In some embodiments, the status of mental health of the subject is determined using an operation different than the operation 205. At an operation 220, the method 200 may comprise providing additional data comprising a determined status of mental health of the subject to a ML tool. At an operation 225, the method 200 may comprise training (or retraining) a ML algorithm. The machine learning algorithm may be a part of the machine learning tool.

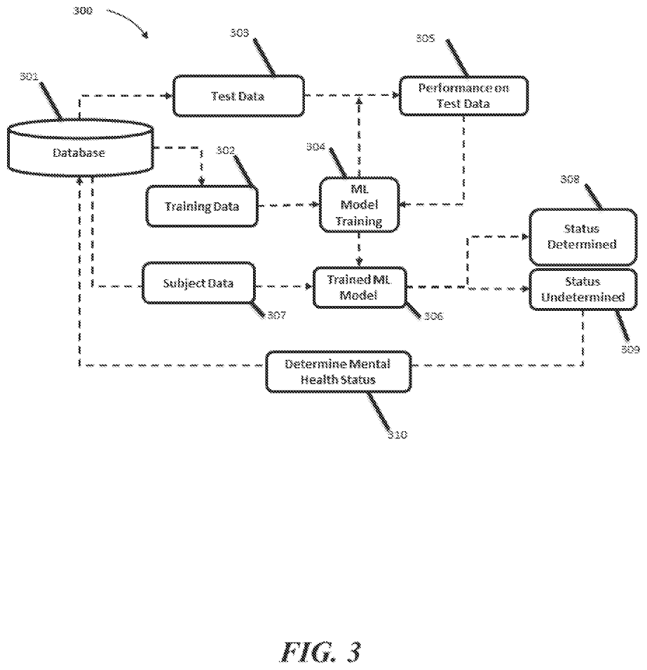

FIG. 3 shows a schematic flow chart of an example machine learning tool (ML tool) 300. The ML tool may comprise a database 301 comprising data attributable to a subject and/or data attributable to a plurality of subjects (e.g., a population comprising subjects with or without a mental health condition comprising a mental health status). The data in the database 301 may be separated into at least two sets comprising at least one training data set 302, and at least one test data set 303. A test dataset may comprise between about 5% to about 50% of the data in the database. In some cases, the test dataset may comprise about 5% to about 15% of the data in the database. In some embodiments, the test dataset and a training data set may be selected from the database substantially randomly. In some embodiments, the ML tool may comprise a ML model 304. The training data set 302 may be used to train the ML model 304 (e.g., machine learning algorithm). At an operation 305, a performance of the ML model 304 may be tested using the test dataset. In some cases, a plurality of training datasets and test datasets may be selected form the database to train and/or test the ML model. A trained ML model 306 may be used to process data attributable to a subject 307 to determine a mental health status of the subject. In some cases, the data attributable to the subject may not be used to train and/or test the ML model. A status of the mental health of the subject may be determined, at operation 308, using the trained ML model. In some cases, the status of mental health of the subject may be undetermined, at operation 309, by the trained ML model. In some cases, the mental health status of the subject undetermined by the trained ML model may be determined by a method other than the trained ML model (e.g., a clinical test). In some cases, the mental health status of the subject, at operation 310, determined by a method other than the trained machine learning model may be added to the database and/or used to train and/or test an ML algorithm (e.g., ML model 304).

In some cases, the machine learning tool may comprise at least a machine learning algorithm and a database. For example, the machine learning algorithm may comprise one or more of: linear regression, logistic regression, classification and regression tree algorithm, support vector machine (SVM), naive Bayes, K-nearest neighbor, random forest algorithm, boosted algorithm such as XGBoost and LightGBM, neural network, convolutional neural network, and recurrent neural network. In some cases, the machine learning algorithm may comprise a Gradient Boosting Decision Tree (GBDT) model. The GBDT model may be used for non-linear data. The machine learning algorithm may be a supervised learning algorithm, an unsupervised learning algorithm, or a semi-supervised learning algorithm.

In some cases, a nested model is used to evaluate a mental health status of a subject by assessing key markers of the subject's health. The model may comprise generating a risk score for perinatal mood and anxiety disorders, such as perinatal depression. In some cases, the risk score is generated periodically (e.g., weekly, biweekly, monthly, etc.). In some cases, the risk score is generated almost on demand (e.g., requested by a healthcare provider). In some cases, the model comprises generating a recommendation. The recommendation may be a personalized recommendation. The recommendation may be an action according to a standard of care.

In some cases, the machine learning algorithm determines a trend over time. The trend may be a trend of a mental health disorder. A trend may be a behavioral trend (e.g., exercising habits, eating behavior, sleeping patterns, changes in speech). For example, to determine a mental health status of a subject, the ML algorithm compares a pattern of the subject (e.g., a behavior pattern) to a baseline pattern from the same subject, and/or to a pattern from other subjects. The other subjects may or may not have been diagnosed with a mental health disorder. The other subjects may have been diagnosed for one or more health issues. For example, in order to predict a status of a subject as perinatal depression (e.g., prenatal or postpartum depression), the machine learning algorithm captures mental health states at various stages in perinatal period. The ML algorithm may then determine a pattern in the captured data. The captured data may then be compared to a pattern determined from captured data from a plurality of subjects that may or may not have been diagnosed with depression. A pattern (or trend) may be identified as normal or alarming based at least in part on the comparison of the determined patterns. In some cases, a sequence model is used to quantify an impact of a change in a pattern on the accuracy of the ML algorithm.

In some cases, a mental health status of a subject may be undetermined. The undetermined mental health status of the subject may comprise a status that may correspond to lower than, substantially close to, or at a screening or diagnostic (e.g., depression diagnostic) threshold (e.g., a threshold of a presence or absence of a mental disorder according to clinical guidelines) of a diagnosis tool, such as, for example, the Edinburgh Postnatal Depression Scale (EPDS), Patient Health Questionnaire (PHQ) or General Anxiety Disorder (GAD) screening tool. In some cases, subject has a mental state corresponding to within 1%, within 2%, within 3%, within 4%, within 5%, within 6%, within 7%, within 8%, within 9%, within 10%, within 15%, within 20%, within 25%, within 30%, within 40%, within 50%, or more above or below a screening or diagnostic (e.g., depression diagnostic) threshold value on the Edinburgh Postnatal Depression Scale (EPDS), Patient Health Questionnaire (PHQ) or General Anxiety Disorder (GAD) screening tool.

In some cases, the subject may be above such a threshold. For example, a status of mental health corresponding to a level of slightly lower than (e.g., about 10%-20% lower), slightly higher than (e.g., about 10%-20% higher), or equal to a depression-threshold on the Edinburgh Postnatal Depression Scale (EPDS) may be considered as undetermined. The threshold may be a threshold of a presence of a disorder in a subject. The threshold may be a threshold of an absence of a disorder in a subject. In some cases, a status of mental health of a subject may be determined with a lower confidence level based at least in part on the provided data attributable to the subject. Subsequently, a mental health of the subject may be determined by other methods (e.g., referring a subject to a clinician or by re-determining the status after collecting more data).

In some cases, the status of the mental health of a subject may be determined based at least in part on a test comprising a score. The test may have a predefined threshold for the score to determine a mental health status. The subject may have a score that may be closer than a predefined margin to the threshold (e.g., where a perinatal subject score of 9 in an EPDS questionnaire, where threshold is 10). A score or a threshold may be generated by the model (e.g., the ML algorithm), described herein. In order to improve the accuracy of the ML algorithm to determine the mental health status of the subject, the ML model may be rendered undetermined.

In some cases, the status of the mental health of a subject is determined. The mental health of a subject may be determined by a test, a clinical test, a clinician, etc. For example, a report may be provided to a recipient, where the report comprises a message for an undetermined mental health of the subject. In some cases, the recipient may receive a recommendation comprising subjecting a subject to a mental health evaluation, referring the subject to a mental health provider, etc. subsequently, the recipient may facilitate determining a mental health status of the subject.

In some cases, the determined status may be requested via a request sent to a recipient. For example, an inquiry may be generated (e.g., a personalized inquiry) to request the determined status of mental health of said subject and/or information associated with the determination of the status. In some cases, additional data attributable to the subject associated with the determined status of the mental health of the subject may be collected.

In some cases, an identifier may be generated using the determined mental health status of the subject and/or data associated with the determined status (e.g., answer to the personalized inquiry or data collected). The identifier may comprise data associated with the determined status of mental health of the subject. For example, the identifier may comprise an answer to an inquiry and/or data collected from devices, as described hereinbefore. In some cases, the identifier may be added to the database of the machine learning tool.

In some cases, the machine learning algorithm of the machine learning tool may be trained using the identifier comprising the provided status of mental health of the subject and/or data associated with the provided status (e.g., answer to the personalized inquiry or data collected). For example, the machine learning algorithm may be trained by comparing a prediction made using the machine learning model to the identifier comprising a provided status of mental health of a subject determined by methods other than the machine learning model. In some cases, the provided status of mental health of a subject was determined before an identifier or data attributable to a subject was subjected to a machine learning model. The machine learning tool may be trained using a data attributable to a subject, where a mental health status of a subject may be known.

In some cases, the ML training may continue until the predicted status meets a convergence condition. A convergence condition may comprise an improvement in an accuracy of predicting a mental health status. An improvement in the accuracy may comprise obtaining a small magnitude of an error (e.g., accuracy) in determining a status of health of a subject. For example, the magnitude of an error can be calculated by comparing a predicted status of mental health of a subject with an identifier or a determined (or provided) status of mental health attributable to the subject. In some cases, a convergent condition may be met when a status predicted by the trained machine learning algorithm is substantially similar or the same as the provided status. The improvement in the accuracy may be measured by comparing a prediction of a subject's mental health status using the machine learning algorithm before training a machine learning tool and a predicted status of health of the subject made by the trained machine learning tool. In some cases, by training a machine learning tool the accuracy may improve by at least 80%. In some cases, the accuracy may improve by about 50% to about 99%. In some cases, the accuracy may improve by about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 99%. In some cases, the accuracy may improve by about 50%, about 60%, about 70%, about 80%, about 90%, about 99%, about 100%, about 200%, about 300%, about 400%, about 500%, about 1000% or more. In some cases, the accuracy may improve by at least about 50%, about 60%, about 70%, about 80%, about 90%, about 99%, about 100%, about 200%, about 300%, about 400%, about 500%, about 1000% or more. In some cases, the accuracy may improve by at most about 99%, about 90%, about 80%, about 70%, about 60% or less.

A machine learning tool may comprise a machine learning algorithm trained model. In some embodiments, a machine learning tool comprising a machine learning algorithm trained model may be used to assess an individual mental health status in an individual.

The machine learning tool may comprise a machine learning algorithm and a data base. The machine learning algorithm may comprise a supervised learning approach. The database may comprise a training data. In supervised learning, the algorithm can generate a function or model from a training data. The training data can be labeled. The training data may include metadata associated therewith. Each training example of the training data may be a pair consisting of at least an input object and an appropriate output value. A supervised learning algorithm may require the user to determine one or more control parameters. These parameters can be adjusted by optimizing performance on a subset, for example, a validation set, of the training data. After parameter adjustment and ML training, the performance of the trained ML can be measured on a test set that may be separate from the training set. Regression methods can be used in supervised learning approaches. In some embodiments, the trained machine learning tool may comprise a classifier model, or a gradient boost decision tree (GBDT) model.

The machine learning (ML) (e.g., machine learning algorithm, machine learning model, machine learning tool) may be configured to accept a plurality of input variables and to produce one or more output values based on the plurality of input variables. The plurality of input variables may comprise data attributable to a subject or a plurality of subjects collected automatically or by an inquiry. For example, an input variable may comprise a set of data associated with social data, behavioral data, biological data, affective or cognitive (e.g., neurocognitive) data, experiential data, psychomotor activity data, expressive behavioral data, sociodemographic data, medical data or other health marker data.

The ML may have one or more possible output values, each comprising one of a fixed number of possible values indicating a status of mental health. The output value of an ML may comprise discrete value. An ML output value may comprise one of two or more potential values. For example, an output value may be one of two values (e.g., a presence or an absence of a condition, a 0 or 1, a positive or a negative value). The output value may indicate a classification of the mental health status (e.g., level of depression, severity of perinatal depression). The output values may comprise more than two values. For example, a presence of a condition, an absence of a condition, or an undetermined condition (e.g., no depression present, depression present, or status of depression is undetermined). A value may indicate a severity of a condition, for example, a very low, a low, a medium, a high, and/or a very high severity of a mental health condition. Some of the output values may comprise descriptive labels. Such descriptive labels may provide an identification or indication of the mental health status (e.g., level of mental health status) of the subject, and may comprise, for example, positive status, low negative status, medium negative status, high negative status, and/or very high negative status. For example, an output may be selected from a list of values including depressed, not depressed, highly depressed indicating a classification of the mental health status. A negative status may comprise a mental health disorder or condition. Such descriptive labels may provide an identification of a recommendation for the subject's mental health status (e.g., to improve a negative status, or maintain a positive status), and may comprise, for example, a therapeutic intervention, a duration of the therapeutic intervention, and/or a recommendation related to diet, exercise, sports training, supplements, functional tests, blood tests, brain management, behavior change, social support structure, environmental exposure, stress management, and/or mental health. Such descriptive labels may provide an identification of secondary clinical tests that may be appropriate to perform on the subject, and may comprise, for example, a biopsy, a blood test, a saliva test, a functional test, a computed tomography (CT) scan, a magnetic resonance imaging (MRI) scan, an ultrasound scan, a chest X-ray, a positron emission tomography (PET) scan, or a PET-CT scan. Such descriptive labels may provide a prognosis of the disease state of the subject. Some descriptive labels may be mapped to numerical values, for example, by mapping “positive” to 1 and “negative” to 0.

The output value of an ML may comprise a continuous (or concrete) output value. An output may comprise, for example, a probability value of at least 0 and no more than 1, or a percentage between 0% to 100% (e.g., of the probability of the mental health status of a subject). The continuous output may be normalized based on a baseline value or may be un-normalized. A threshold value may be assigned to a continuous ML output values. For example, a threshold of a probability of a mental health status in a subject may comprise one or more numbers between 0 to 1 or a number between 0% to 100%. There may be more than one threshold in the output values that may indicate a probability of higher or lower severity of a mental health status in a subject. For example, an ML may predict a status of mental health of a subject to be at least a 50% probability indicating that there may be a need for an intervention as a result of a mental health status (e.g., a negative status, a perinatal depression, or anxiety). For example, a probability of less than 50% may indicate an absence of a mental health status in a subject. In some cases, the threshold may comprise a continuous range. For example, a probability of a subject having a mental health status between a first output value (e.g., about 40%) to a second output value (e.g., about 60%) from the output values may be considered an undetermined status, while a value below the first output value may indicate an absence of a status and/or an output value above a second value may indicate a presence of a mental health status. An ML may use a threshold to generate a binary classification. For example, above a threshold or below a threshold may correspond to a binary classification of a status of mental health of a subject. A binary classification of a status of mental health may assign an output value of “negative” or 0 if the data indicate that the subject has less than a 50% probability of being recommended an intervention as a result of a mental health status. In this case, a single threshold value of 50% is used to classify the status of mental health of a subject into one of the two possible binary output values. Examples of single threshold values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, and about 99%.

The ML may be trained with a plurality of independent training datasets. Each of the independent training datasets may comprise collected data from inquiries or data collected automatically from a subject, associated data obtained by processing the collected data, and one or more known output values corresponding to mental health status of a subject. Independent training datasets may comprise collected data from inquiries or data collected automatically from a plurality of different subjects. Independent training datasets may comprise collected data from inquiries or data collected automatically obtained at a plurality of different time points from the same subject. Independent training datasets may be associated with presence of a mental health status (e.g., comprise collected data from inquiries, data collected automatically, or associated data obtained by processing the data collected from a plurality of subjects known to have a mental disorder such as a perinatal depression). Independent training datasets may be associated with absence of a mental health status (e.g., comprise collected data from inquiries, data collected automatically, or associated data obtained by processing the data collected from a plurality of subjects known to not have a mental disorder such as a perinatal depression).

The ML may be trained with at most about 500, at most about 400, at most about 200, at most about 100, at most about 50, at most about 30, at most about 20, at most about 10, at most about 8, at most about 7, at most about 6, at most about 5, at most about 4, at most about 3, at most about 2, at most about 1 independent training datasets. The independent training datasets may comprise data associated with presence of a mental health disorder (e.g., depression) and/or data associated with absence of a mental health disorder (e.g., depression). The ML may be trained with at least 1, 2, 3, 4, 5, 6, 7, 8, 9, 10, 20, 50, 100, 200, 300, 400, 500, 600, or more independent training datasets associated with presence of a mental health disorder (e.g., depression). In some embodiments, the training dataset data is independent of data attributable to a subject or plurality of the subjects used to train the ML. For example, an identifier can be generated that identifies the data attributable to the subject as attributable to the mental status of the subject. This identifier may be independent from the data attributable to a subject or plurality of the subjects used to train the ML.

The ML may be trained with a first number of independent training datasets associated with a presence of a mental health disorder (e.g., depression) and a second number of independent training datasets associated with an absence of a mental health disorder (e.g., depression). The first number of independent training datasets associated with a presence of a mental health disorder (e.g., depression) may be no more than the second number of independent training datasets associated with an absence of a mental health disorder (e.g., depression). The first number of independent training datasets associated with a presence of a mental health disorder (e.g., depression) may be equal to the second number of independent training datasets associated with an absence of a mental health disorder (e.g., depression). The first number of independent training datasets associated with a presence of a mental health disorder (e.g., depression) may be greater than the second number of independent training datasets associated with an absence of a mental health disorder (e.g., depression).

An accuracy of identifying a status of mental health of a subject by the ML may be calculated as the percentage of independent test datasets (e.g., subjects having a mental health disorder) that are correctly identified or classified as having or not having the mental health disorder, respectively. The ML may be configured to identify a status of mental health of a subject with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%; for at least about 50, at least about 100, at least about 150, at least about 200, at least about 250, at least about 300, or more than about 300 independent datasets. For example, the accuracy can be calculated as the percentage of subjects diagnosed for perinatal depression that were correctly identified by ML to have a perinatal depression. A positive predictive value (PPV) of identifying a status of mental health by the ML may be calculated as the percentage of subjects identified or classified as having a mental health disorder or condition (e.g., perinatal depression) that correspond to subjects that truly have that mental health disorder or condition (e.g., for example as confirmed by clinical diagnosis). A PPV may also be referred to as a precision. The ML may be configured to identify a status of mental health with a PPV of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%.

A negative predictive value (NPV) of identifying a status of mental health by the ML may be calculated as the percentage of subjects identified or classified as not having a mental health disorder or condition (e.g., perinatal depression) that correspond to subjects that truly do not have that mental health disorder or condition (e.g., perinatal depression). The ML may be configured to identify a status of mental health with an NVP of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%.

A sensitivity of identifying a status of mental health by the ML may be calculated as the percentage of independent subjects with presence of a mental health disorder or condition (e.g., perinatal depression) that are correctly identified or classified as having that mental health disorder or condition (e.g., perinatal depression). The ML may be configured to identify a status of mental health with a sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. A sensitivity may also be referred to as a recall.

A specificity of identifying a status of mental health by the ML may be calculated as the percentage of independent subjects with an absence of a mental health disorder or condition (e.g., apparently healthy subjects with negative clinical diagnosis for a mental health disorders) that are correctly identified or classified as not having that mental health disorder or condition. The ML may be configured to identify a status of mental health with a specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%.

An Area-Under-Curve (AUC) may be calculated as an integral of the Receiver Operator Characteristic (ROC) curve (e.g., the area under the ROC curve) associated with the ML in classifying or determining a status of mental health in a subject as having or not having that mental health status. The ML may be configured to identify a status of mental health with an AUC of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

The ML may be adjusted or tuned to improve the performance, accuracy, PPV, NPV, sensitivity, specificity, or AUC of determining a mental health status of a subject. The ML may be adjusted or tuned by adjusting parameters of the ML (e.g., a set of threshold values used to determine a mental health status of a subject as described elsewhere herein, or weights of a neural network). The ML may be adjusted or tuned substantially continuously during the training process or after the training process has completed.

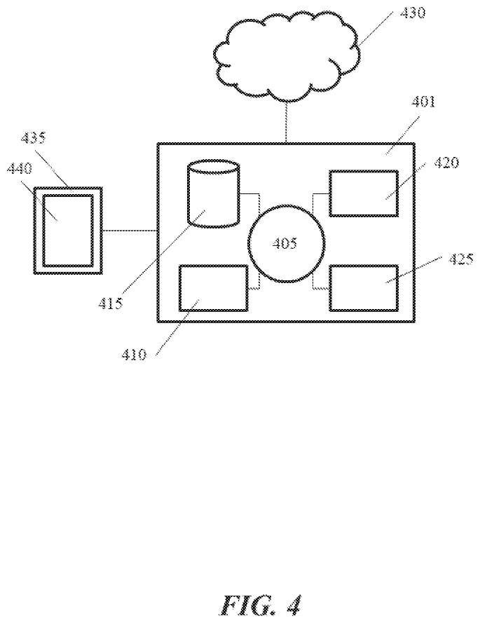

The present disclosure provides computer systems that are programmed to implement methods of the disclosure. FIG. 4 shows a computer system 401 that is programmed or otherwise configured to perform methods described herein. In some cases, the computer system can be configured to collect data attributable to a subject or a plurality of subjects, provide the data to a machine learning model (e.g., ML algorithm or a ML tool), determine a mental health status of the subject, build or retrain a ML tool, communicating with a subject, or provide the determined status to a recipient. The computer system 401 can regulate various aspects of the present disclosure, such as, for example, time and period of collecting data, frequency of processing data, building a ML tool, and/or time of and/or frequency of providing data to a recipient. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., a mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, an inquiry, a questionnaire, a status of a mental health of a subject to a recipient or a subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, collect data attributable to a subject or a plurality of subjects, provide the data to a machine learning model (e.g., ML algorithm or a ML tool), determine a mental health status of the subject, determine if a status of mental health of a subject is undetermined to send an inquiry to determine the status, use the determined status to build or retrain a ML tool, communicating with a subject, or provide the determined status to a recipient.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for monitoring mental health of a subject, comprising:
(a) collecting data attributable to said subject at different time points, wherein said data is derived from answers to inquiries provided on a plurality of requests individualized to said subject;
(b) providing said data to a computer system programmed with a machine learning algorithm, which machine learning algorithm processes said data of plurality of time-points and determines a status of mental health of said subject; and
(c) providing said status of mental health of said subject to said subject or other recipient(s).

2. The method of claim 1, wherein said subject is pregnant or postpartum.

3. The method of claim 1, wherein said subject is not perinatal.

4. The method of claim 1, wherein said status of mental health comprises a status of depression, a status of anxiety, a status of mood, a status of obsessive compulsive disorder, a status of psychosis, a status of suicidality, a status of distress, a status of stress, a status of bipolar disorder, a status of baby blues, a status of post-traumatic stress disorder, a status of eating disorder, a status of sleep disorder or any combination thereof.

5. The method of claim 4, wherein said status of mental health comprises a status of perinatal-associated mental health disorder or perinatal mood and anxiety disorder.

6. The method of claim 1, wherein said machine learning algorithm processes said data and determines a risk of a mental condition in said subject.

7. The method of claim 6, wherein said machine learning algorithm determines a risk score for said mental condition in said subject.

8. The method of claim 1, wherein said mental health status is predictive.

9. The method of claim 1, further comprising providing a recommendation associated with said status of mental health of said subject to said recipient and wherein said recommendation comprises a recommendation for a therapy or sources of education associated with said status of mental health.

10. The method of claim 1, further comprising alerting said recipient to a behavioral risk associated with said status of said mental health.

11. The method of claim 10, wherein said behavioral risk is a risk of suicide of said subject, risk of infanticide being committed by said subject, risk of developing depression, risk of developing anxiety, risk of developing obsessive compulsive disorder, risk of developing psychosis, risk of developing distress, risk of developing stress, risk of developing bipolar disorder, risk of developing baby blues, risk of developing post-traumatic stress disorder, risk of developing a sleep disorder, risk of developing an eating disorder or any combination thereof.

12. The method of claim 11, wherein said behavioral risk is a perinatal behavioral risk.

13. The method of claim 1, wherein said answers to said inquiries are provided by said subject.

14. The method of claim 1, wherein said answers to said inquiries are not provided by said subject.

15. The method of claim 1, wherein said answers to said inquiries are provided via automatic data extraction.

16. The method of claim 1, wherein at least two requests of said plurality of requests comprise at least one different inquiry or wherein a request of said plurality of requests comprises a single inquiry.

17. The method of claim 1, wherein at least one request of said plurality of requests is individualized to said subject based on answers to another request that precedes said at least one request.

18. The method of claim 1, wherein (a) further comprises collecting said data attributable to said subject over a time period of at least two weeks.

19. The method of claim 1, wherein said inquiries include an inquiry associated with health, an inquiry associated with weight, an inquiry associated with social interactions, an inquiry associated with a physiologic state, an inquiry associated with cognitive affective state, an inquiry associated with environmental conditions, an inquiry associated with healthcare utilization, an inquiry associated with mood, an inquiry associated with exercise level, an inquiry associated with nutrition, an inquiry associated with appetite, an inquiry associated with tobacco usage, an inquiry associated with alcohol consumption, an inquiry associated with social support, an inquiry associated with expressive behavior, an inquiry associated with psychomotor activity, an inquiry associated with sleep, an inquiry associated with social and behavioral markers of health, an inquiry associated with depression, an inquiry associated with anxiety, an inquiry associated with distress, an inquiry associated with stress, an inquiry associated with obsessive compulsive behavior, an inquiry associated with psychosis, an inquiry associated with suicidality, an inquiry associated with daily experiences, an inquiry associated with childcare, an inquiry associated with breastfeeding, an inquiry associated with parenting, an inquiry associated with prior mental health problems, an inquiry associated with medical history, an inquiry associated with familial medical history, an inquiry associated with substance abuse, an inquiry associated with clinical diagnosis, an inquiry associated with socio-demographic state, an inquiry associated with family structure, an inquiry associated with household conditions, an inquiry associated with exposure to domestic violence or sexual assault, an inquiry associated with living conditions, an inquiry associated with subject characteristics, an inquiry associated with education, an inquiry associated with income level, or any combination thereof.

20. The method of claim 1, further comprising collecting additional data attributable to said subject (i) via a device configured to monitor one or more health or wellness markers associated with said subject or (ii) from an individual.

21. The method of claim 20, wherein said device is a mobile electronic device.

22. The method of claim 20, wherein said individual is a household member of said subject, a parent of said subject, a friend of said subject or a care-provider.

23. The method of claim 22, wherein said care-provider is a health-care provider, a lactation consultant, a psychotherapist, a psychiatrist, a physical therapist, a health and wellness provider or a doula.

24. The method of claim 22, wherein said one or more health or wellness markers is sleep, an activity level, an exercise level, nutrition, appetite, weight, an emotional state, social relations, psychomotor activity, expressive behaviors, a bonding of said subject with said subject's children, or any combination thereof.

25. The method of claim 20, further comprising providing said additional data to said machine learning algorithm which machine learning algorithm processes said additional data to determine said status of mental health of said subject.

26. The method of claim 1, further comprising collecting additional data obtained from one or more medical or clinical tests conducted with respect to said subject.

27. The method of claim 26, wherein said one or more medical tests comprise a blood test, a saliva test, a screening test, a clinical diagnostic test, a test under Diagnostic and Statistical Manual of Mental Disorders (DSM) guidelines, a biometric test, an activity test, a sleep test, a mental health test, psychoanalysis or a behavioral test.

28. The method of claim 1, further comprising collecting additional data obtained from one or more medical or clinical diagnosis made with respect to said subject.

29. The method of claim 1, wherein, in (a), said status of said mental health of said subject is unknown.

30. The method of claim 1, wherein said subject has a mental state corresponding to within 10% above or below a screening or diagnostic-threshold value on the Edinburgh Postnatal Depression Scale (EPDS), Patient Health Questionnaire (PHQ) or General Anxiety Disorder (GAD) screening tool.
